(19) United States
(12) Patent Application Publication
(10) Pub. No.: US 2008/0096947 A1
(43) Pub. Date: Apr. 24, 2008

(54) Parenteral Forms Of Administration Of Imexon And Method For The Production Thereof

(75) Inventors: Hans-Georg Opitz, Weinheim (DE); Heinrich J. Woog, Laudenbach (DE); Gruber Werner, Birkenau (DE)

Correspondence Address: FOLEY AND LARDNER LLP;SUITE 500, 3000 K STREET NW, WASHINGTON, DC, 20007, US

(73) Assignee: Heidelberg Pharma AG

(21) Appl. No.: 11/664,513

(22) PCT Filed: Oct. 11, 2005
(86) PCT No.: PCT/EP05/10913

§ 371 Date: May 21, 2007

(30) Foreign Application Priority Data
Oct. 15, 2004 (EP) 04024606.8

(51) Int. Cl. A61K 31/4164; A61P 35/00; C07D 487/04

(52) U.S. Cl. 514/387; 548/302.7

(57) ABSTRACT

The invention relates to intravenously highly compatible imexon injection preparations and processes for their production as well as imexon lyophilisates stable in storage for the production of the injection preparations.

The present invention relates to intravenously highly compatible imexon injection preparations and processes for their production as well as imexon lyophilisates stable in storage for the production of the injection preparations.

Imexon (4-imino-1,3-diazabicyclo-(3.1.0)-hexane-2-one) is an aziridine derivative which has attracted attention in the field of pharmacology in that it exerts a preferential cytostatic effect on B cells of the immune system. It is used above all for the therapy of B cell or plasma cell neoplasiae, autoimmune diseases, lymphatic lymphomas and rejection reactions after tissue or organ transplants.

The production and use of imexon and its physiologically compatible salts are described in U.S. Pat. No. 4,083,987 and U.S. Pat. No. 5,369,119.

Imexon has a low bioavailability such that therapeutically effective dosages can be administered only intravenously. These dosages amount to between 50 mg and 1000 mg per day, preferably 200-500 mg per day. The production of an aqueous parenteral galenic formulation of imexon, as described in U.S. Pat. No. 4,083,987, has, however, proved to be a problem. Although imexon is highly soluble in water, it is not stable. When the aziridine ring is opened, it isomerises to products which are insoluble in water and which precipitate.

The requirements regarding an active principle formulation to be administered intravenously or subcutaneously are generally as follows:
a chemical composition which does not change, i.e. no decomposition of the active principle on prolonged storage over several years
sterility
freedom from pyrogen
isotony
good vein or tissue compatibility

Good vein and tissue compatibility of injection solutions depends in turn on the following factors:
the pH (as close to the pH of blood as possible, i.e. between 6.8 and 7.2)
a low buffer capacity
a low titration basicity or low titration acidity.

The latter should be understood to mean the quantity of 1N acid or 1N base which is required in order to increase the quantity of injection solution to be injected to the pH of blood of 6.8-7.2.

As mentioned above, imexon decomposes in aqueous solutions after a short period. The same result is obtained if imexon is lyophilised from aqueous solutions.

Lyophilisation or freeze drying is a process in which aqueous solutions of substances, among others also of pharmaceutical active principles, are usually frozen and the water is removed by sublimation under vacuum as well as by slight heating. In order for the lyophilisate to be highly soluble for further use as injection solution, it should form a so-called cake with the active principle. This has a large surface area and can thus be rapidly dissolved in a suitable solvent. For this reason, suitable builders are usually added to the active principle concentrate provided for lyophilisation. These consist of e.g. manitol, sorbitol, milk sugar, urea, dextran, cyclodextrin, amino acids, polyvinyl pyrrolidone (PVP) and others. In addition, surface-active substances (wetting agents) such as polysorbates (e.g. Tween20, Tween80 from Sigma Aldrich) are capable of improving the solubility during the production of the injection solution to be applied.

Surprisingly, it has now been found that imexon remains stable over a prolonged period in some organic solvents, in contrast to water, and these solvents can consequently be considered in particular for the production of corresponding lyophilisates.

Dimethyl sulphoxide (DMSO) has proved to be a particularly suitable solvent; moreover, solvents such as Solutol HS 15 (Macrogol-15-hydroxystearate), tert. butanol, isobutyl acetate and mixtures of these solvents can be used together. In principle, all physiologically highly compatible solvents having a sufficient vapour pressure are suitable. Moreover, a certain polarity is a precondition for imexon or its salts to dissolve sufficiently.

A precondition for the use of a solvent for the lyophilisation of pharmaceutical active principles is that the solvent does not have a high toxicity level since, as a rule, a solvent residue of approximately 3-5% remains in the lyophilisate after lyophilisation.

Consequently, the problem arose of finding lyophilisate recipes in which imexon or its physiologically compatible salts (e.g. with 25 mg/ml of imexon) are stable in storage for years, which can be dissolved in a highly vein-compatible solvent shortly before application and which are stable in the solution for at least 60 to 120 minutes. Apart from the stability of the active principle, these injection solutions must be free from visible particles and satisfy all the requirements of the European Pharmacopoeia (EP) and United States Pharmacopoeia (USP).

Surprisingly, this problem is solved by lyophilisates containing imexon or its salts, a builder and a wetting agent, lyophilisation taking place from an organic solvent or a mixture of two or more organic solvents. This problem is further solved by way of a process for the production of physiologically compatible imexon lyophilisates stable in storage in the case of which imexon or its salts is/are dissolved together with a builder and a wetting agent in an organic solvent and lyophilised.

In a preferred embodiment, such stable lyophilisate recipes can be obtained if imexon or its salts is/are dissolved together with builders and wetting agents (e.g. PVP (e.g. Kollidon 12PF from BASF) and polysorbate (e.g. Polysorbate 80 from Sigma Aldrich) in pure organic solvents such as e.g. DMSO, the solution is filled through a sterile filter with a pore width of 0.2 μm under aseptic conditions into injection flasks or ampoules and lyophilised. The flasks are subsequently provided with a nitrogen cushion, closed with rubber stoppers and flanged with aluminium caps. Lyophilisates thus produced are stable in storage at 4° C. to 8° C. for several years, i.e. the imexon lyophilisates do not exhibit any active principle decomposition and are present as a voluminous, highly soluble lyophilisate cake. DMSO has proved to be the preferred solvent.

DMSO is a clear colourless and odourless liquid at room temperature whose melting point is situated at around 20° C. It is miscible with water and organic solvents. Although DMSO is hygroscopic, a stable solution of imexon can be produced therewith out decomposition of imexon taking place by opening of the aziridine ring. Although DMSO has a very low vapour pressure of 0.8 hPa (0.6 torr) at 25° C., which can cause long lyophilisation times and large amounts of DMSO residues in the lyophilisate, a stable lyophilisate of imexon can be produced by using DMSO as solvent, the DMSO residue in the lyophilisate being thus limited to 5%. The absolute quantity of DMSO consequently does not exceed the toxicologically liberated daily dose. In order to dissolve the lyophilisates thus obtained within 5 minutes, a 10% by weight aqueous propylene glycol solution was used. In this way, a precipitation of decomposition products of the active principle is prevented and thus a low particle injection solution in line with EP and USP is obtained.

Manitol, sorbitol, milk sugar, urea, dextran, cyclodextrin, amino acids and polyvinyl pyrrolidone are particularly suitable as builders. Polyvinyl pyrrolidone is particularly preferred.

Suitable wetting agents are surface-active substances such as surfactants, polysorbates being particularly preferred.

If desired, the lyophilisates may contain further physiologically compatible substances and substances compatible with imexon and with the builder and the wetting agent, such as e.g. further active principles etc. In a particularly preferred embodiment, no further substances are contained therein.

The lyophilisates according to the invention dissolve after approximately 5 minutes in 10 ml of solvent consisting of:
9 g of water
1 g of propylene glycol

If imexon is used as the salt, this should be understood to mean any physiologically compatible anion, e.g. acetate, citrate, lactate, chloride, bromide, sulphate, phosphate, sulphonate or such like. These salts are obtained by converting imexon which aqueous acids.

Unless indicated otherwise, all data in % relate to the weight.

The following examples are to illustrate the invention in further detail.

### EXAMPLE 1

### Production of a 100 mg/pro Vial Imexon Lyophilisate from Imexon Dissolved in DMSO

| | |
|---|---|
| 1 vial | 1000 vials |

| | | | | |
|---|---|---|---|---|
| Imexon | 100 | mg | 100 | g |
| Kollidon 12 Pf | 30 | mg | 30 | g |
| Polysorbate 80 | 0.002 | mg | 2 | g |
| Dimethyl sulphoxide ad | 4 | g | 4000 | g |

100 g of imexon are dissolved together with 30 g of Kollidon 12 PF and 2 g of Polysorbate 80 in 4000 g of dimethyl sulphoxide, subjected to sterile filtration on a sterile filter with a pore width of 0.2 μm and subsequently filled under aseptic conditions in quantities of 4 g each into sterile injection flasks (10 ml) and introduced into a sterilised lyophilisation facility. The imexon solution is frozen at −40° C. within 3 hours and subsequently dried via the lyophilisation phases of main drying and post-drying for a total of 133 hours.

Subsequently, the lyophilisation chamber is treated with sterile, water-free nitrogen and the flasks are closed with previously freeze-dried stoppers (removal of the water which had penetrated inside during washing of the stoppers) and closed with flanged caps of aluminium.

The lyophilisate thus obtained corresponded to the specifications and criteria of the EP and USP.

### EXAMPLE 2

### Production of an Imexon Lyophilisate of 50 mg/Vial Imexon from Imexon Dissolved in DMSO and Tert. Butanol

| | |
|---|---|
| 1 vial | 1000 vials |

| | | | | |
|---|---|---|---|---|
| Imexon | 50 | mg | 50 | g |
| Kollidon 12 PF | 15 | mg | 15 | g |
| Polysorbate 80 | 0.001 | mg | 1 | g |
| tert. Butanol | 1 | g | 1 | kg |
| Dimethyl sulphoxide ad | 4 | g | 4 | kg |

The production of the lyophilisate takes place in a manner analogous to example 1.

### EXAMPLE 3

### Production of an Imexon Lyophilisate of 50 mg/Vial Active Principle from Imexon Dissolved in Tert. Butanol

| | |
|---|---|
| 1 vial | 1000 vial |

| | | | | |
|---|---|---|---|---|
| Imexon | 50 | mg | 50 | g |
| Kollidon 12 PF | 15 | mg | 15 | g |
| Polysorbate 80 | 0.01 | mg | 1 | g |
| tert. Butanol ad | 4 | g | 4 | kg |

The production of the lyophilisate takes place in a manner analogous to example 1.

### EXAMPLE 4

### Production of a 100 mg/Vial Imexon Lyophilisate with Cyclodextrin as Cake Forming Agent

| | |
|---|---|
| 1 vial | 1000 vials |

| | | | | |
|---|---|---|---|---|
| Imexon | 100 | mg | 100 | g |
| Cyclodextrin | 20 | mg | 20 | g |
| Polysorbate 20 | 0.002 | mg | 2 | g |
| Dimethyl sulphoxide ad | 4 | g | 4000 | g |

The production of the lyophilisate takes place in a manner analogous to example 1.

### EXAMPLE 5

### Production of an Injection Solution from the Lyophilisates According to Example 1 to 4

First, solvent ampoules or vials having the following composition are produced:

| | 1 vial or | 1000 vial or |
|---|---|---|
| | ampoule | ampoules |
| Propylene glycol | 1 g | 1000 g |
| Water for injection purposes | 9 g | 9000 g |

The two solvents are mixed intensively in a vessel, subsequently subjected to sterile filtration in a sterile filter with pore size of 0.2 μm under aseptic conditions and filled by means of an automatic filling machine into 10 ml vials or ampoules and closed.

The filled vials or ampoules are sterilised for 20 minutes at 121° C. in the autoclave.

A 10 ml content of one ampoule or vial each is added to one vial of lyophilisate each and the lyophilisate is dissolved by shaking. The solution thus obtained is sucked up into an injection syringe and injected. In this way, a clear, particle-free, highly compatible injection solution is obtained which remains stable for at least 120 minutes and is suitable for application.

1. Physiologically compatible imexon lyophilisates stable in storage containing the active principle imexon or its salts, a builder and a wetting agent characterised in that they are lyophilised from an organic solvent or a mixture of two or more organic solvents.

2. Imexon lyophilisates according to claim 1, polyvinyl pyrrolidone or cyclodextrin being preferably used as builders.

3. Imexon lyophilisates according to claim 1, a polysorbate being used as vein compatible wetting agent.

4. Lyophilisates according to claim 1, the solvent used for lyophilisation being a physiologically highly compatible organic solvent selected from the group of dimethyl sulphoxide, tert. butanol, Solutol HS 15, isobutyl acetate, ethyl acetate or mixtures of these solvents.

5. Lyophilisates according to claim 1, consisting of imexon or its salts, the builder and the wetting agent as well as up to 5% solvent.

6. Process for the production of physiologically compatible imexon lyophilisates stable in storage containing at least one builder and one wetting agent characterised in that imexon or its salt(s) is/are dissolved together with a builder and a wetting agent in an organic solvent and lyophilised.

7. Process according to claim 6 characterized in that dimethyl sulphoxide is used as the organic solvent.

8. Process according to claim 6 characterized in that tert. butanol, Solutol HS15, isobutyl acetate, ethyl acetate or mixtures of these solvents are used as the solvent.

9. Process for the production of parenteral forms of administration of imexon or its salts characterised in that an imexon lyophilisate according to claim 1, is dissolved for application in a solution of 10% propylene glycol and 90% water.
